(19) United States
(12) Patent Application Publication
(10) Pub. No.: US 2022/0118154 A1
(43) Pub. Date: Apr. 21, 2022

(54) CLAY MINERAL-BASED TREATMENTS IN PSEUDOMONAS AERUGINOSA INFECTION CONTROL

(71) Applicant: OIL-DRI CORPORATION OF AMERICA, Chicago, IL (US)

(72) Inventors: Dongping Wang, Buffalo Grove, IL (US); Hongyu Xue, Hawthorn Woods, IL (US)

(21) Appl. No.: 17/504,338

(22) Filed: Oct. 18, 2021

Related U.S. Application Data
(60) Provisional application No. 63/093,574, filed on Oct. 19, 2020.

(51) Int. Cl. A61L 15/42; A61P 31/04; A01N 37/36

(52) U.S. Cl. CPC A61L 15/42; A01N 37/36; A61P 31/04

(57) ABSTRACT

The present disclosure relates to a method of treating a Pseudomonas aeruginosa infection, comprising: administering an effective amount of thermally activated clay, wherein the thermally activated clay absorbs pyocyanin and siderophore pyoverdine secreted by Pseudomonas aeruginosa as opposed to exhibiting antibiotic effects such as inhibition of microbial growth or outright killing the bacteria.

### RELATED APPLICATIONS AND INCORPORATION BY REFERENCE

This application claims benefit of and priority to U.S. provisional patent application Ser. No. 63/093,574 filed Oct. 19, 2020. The foregoing application, and all documents cited therein or during its prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the disclosure. More specifically, all referenced documents are incorporated by reference to the same extent as if each individual document was specifically and individually indicated to be incorporated by reference.

### FIELD OF THE DISCLOSURE

The present disclosure generally relates to a composition and method of treating Pseudomonas aeruginosa infection with clay minerals. More specifically, the present disclosure is a thermally activated clay which regulates the expression of virulence genes and exerts minimal selection pressure for antimicrobial resistance.

### BACKGROUND OF THE DISCLOSURE

Pseudomonas aeruginosa is a Gram-negative opportunistic bacterium that causes various infections. Common community-acquired infections with P. aeruginosa are skin and soft tissue infections, ulcerative keratitis and otitis externa, while hospital-acquired infections include bloodstream infections, pneumonias, and urinary tract infections. Infections may be associated with a high rate of morbidity and mortality in immunocompromised hosts, such as those suffering from chemotherapy-induced neutropenia, patients with cystic fibrosis or severe burns and individuals who receive intensive care. Recent studies indicate that P. aeruginosa is one of the top ten most frequently occurring pathogen, the second most common cause of ventilator-associated pneumonia and the seventh most common cause of catheter-related bloodstream infection.

Although antibiotics may be used to control the population of P. aeruginosa, such usage usually poses several problems in terms of the generation of resistance in pathogens, overuse of the drugs, and the inadvertent killing of “good” bacteria (thereby reducing bacterial biodiversity in the environment). The use of antibiotics also collaterally impacts the environment; for example, the antibiotics continue to kill bacteria after the initial intended located as it enters waterways and soil and the bacteria themselves leave toxic byproducts when they are killed by the antibiotics. Thus there are advantages to finding an alternative way to control the bacterial population, or the virulence and biofilm formation of them, through the administration of agents that regulates the expression of bacterial virulence genes rather than using antibiotics or other drugs for mas bacterial elimination.

Rates of antibiotic resistant Gram-negative infections continue to rise worldwide, and effective therapeutic options against these infections are severely limited. Each year in Europe, approximately 400,000 patients with hospital-acquired infections present with a resistant strain. Resistance is a particular problem with P. aeruginosa, because of the low permeability of its cell wall and its ability to acquire and express multiple resistance mechanisms including porin deletions and overexpression of efflux pumps. While the prevalence of P. aeruginosa in the last two decades has remained stable, the prevalence of resistant strains has increased dramatically. Resistant P. aeruginosa infections are associated with high mortality, morbidity, and increased resource utilization and costs.

The literature reports compositions comprising clays as being useful in treating diseases in animals by adsorbing toxins or in treating diseases. For example, WO 2010/028215 describes a modified fish food comprising a fish or shrimp fed material; an acidulant; and a clay material, which is reported to be effective in adsorbing aflatoxins. US 2011/0033576 describes compositions comprising yeast cell and/or yeast cell components with an altered cell wall structure (e.g., a clay or clay component interlaced into the cell wall) to sequester bacteria and toxins. US 2014/0099373 provides for methods of treating enteric disease such as those caused by Clostridium bacteria in an animal which comprises administering a mixture comprising a clay, a yeast, a yeast product or a yeast-like product to the animal. US 2016/0030475 provides for treating early mortality syndrome/acute hepatopancreatic necrosis disease in an animal in need thereof by administer a clay or a clay blend to an animal. None of these above-mentioned publications discusses using thermally activated clay to regulate expression of bacterial virulence genes rather than using antibiotics or other drugs for mas bacterial elimination.

Thus, there exists a currently unmet need for an alternative, non-antibiotic treatment for P. aeruginosa.

Citation or identification of any document in this application is not an admission that such document is available as prior art to the present invention.

### SUMMARY OF THE DISCLOSURE

The present disclosure is directed to phyllosilicates, commonly referred to as clay minerals, capable of regulating the expression of both pyocyanin and pyoverdine virulence genes. The phyllosilicates/clay minerals having antivirulence properties which directly target bacterial toxins as well as the expression of virulence genes. The phyllosilicates directly targets the bacterial toxins and the expression of multiple virulence genes and can be used for both preventative care and post-infection treatment by preventing the production of virulence factors and exerting minimal selection pressure for antimicrobial resistance. Further, the present disclosure also relates to a method of administering the phyllosilicates/clay minerals for better health and performance in animals or better health in humans.

In one aspect of the present disclosure, a clay material is mechanically processed and thermally treated, changing the structure of the clay which then allows for the adsorption of toxins onto the mineral surface of the clay. The thermally activated clay is then ground to a fine particle size and then administered at a ratio necessary to regulate the expression of virulence genes or to interfere with the virulence of the pathogen.

Specifically, the clay material is processed and thermally treated at a temperature between 100 to about 800° C. and ground to a fine particle size (between about 10 microns to as large as about 800 microns) (“thermally activated clay”). Thermal activation changes the physical structure and surface properties of the clay minerals, such as porosity, hydrophobicity and the adsorption sites. These changes permit the infiltration of aqueous fluids into the pores, which then allows for the adsorption of toxins into the thermally activate clay mineral surface. Non-limiting examples of clay minerals (phyllosilicates) which may be processed are smectites or bentonites (which include montmorillonite, nontronite, beidellite and saponite); alumino-silicates, sepiolite, attapulgite (palygorskite); kaolins; fuller's earths and other similar compositions.

In another aspect, the formulation is configured to be applied topically to a user. In addition, the composition may be ingested by a user. Also, the composition may be configured to absorb pyocyanin and siderophore pyoverdine secreted by P. aeruginosa. Further, the thermally activated clay may be a processed montmorillonite clay.

In yet another aspect of the present disclosure, a method of treating a P. aeruginosa infection is provided, comprising administering an effective amount of thermally activated clay, wherein the thermally activated clay absorbs and adsorbs pyocyanin and siderophore pyoverdine secreted by P. aeruginosa.

During the course of an infection by P. aeruginosa, pyocyanin and pyoverdine are secreted to aid bacterial colonization and persistence in the hosts. Management of the infection relies on inhibition or removal of these two toxins. After processing, the thermally activated clay minerals harbor the external basal surfaces, edges, and interlayer space that are possible adsorption sites for pyocyanin and pyoverdine. When the thermally activated clay is added to the infection sites, the aqueous fluids containing the two toxins are actively adsorbed into the macropore structures within the minerals. The toxins harbor electron rich functional groups such as keto and amine residues. Once the toxins have been adsorbed by the thermally activated clay, the electron rich functional groups located in the toxins can access the positively charged sites of the thermally activated clay and form a stable complex by chelating with cations or metal sites on the clay surface. The adsorption of the toxins into the porous space of the thermally activated clay physically separates the toxins from the P. aeruginosa infection. Once the P. aeruginosa is deprived of these toxins, the infection is impaired in its ability to take in nutrients or damage the host cell.

In another aspect of the method, the thermally activated clay may be administered topically. In another aspect, the thermally activated clay may be administered topically as a cream onto a burn. Also, the thermally activated clay may be administered enterally. Further, the thermally activated clay formulation may be administered as an aqueous solution containing at least 0.25% by weight of the thermally activated clay. In addition, the thermally activated clay may be a processed montmorillonite clay.

Accordingly, it is an object of the disclosure to not encompass within the disclosure any previously known product, process of making the product, method of using the product, or method of treatment such that Applicants reserve the right and hereby disclose a disclaimer of any previously known product, process, or method. It is further noted that the disclosure does not intend to encompass within the scope of the disclosure any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the disclosure. In addition, the term “thermally activated clay” describes the clay or clay minerals such as smectites or bentonites (which include montmorillonite, nontronite, beidellite and saponite); alumino-silicates, sepiolite, attapulgite (palygorskite); kaolins; and other fuller's earths which have been processed and thermally heat treated between 100 and 800 degrees Celsius.

These and other embodiments are disclosed or are obvious from and encompassed by, the following Detailed Description.

### BRIEF DESCRIPTION OF THE DRAWINGS

The following detailed description, given by way of example, but not intended to limit the disclosure solely to the specific embodiments described, may best be understood in conjunction with the accompanying drawings.

FIG. 1 is an image summarizing the results of an in vitro experiment in accordance with the present disclosure.

FIG. 2a is an image of the line structures for phenazine and pyocyanin.

FIG. 2b is an image summarizing the results of an in vitro experiment in accordance with the present disclosure.

FIG. 3 is an image summarizing the results of an in vitro experiment in accordance with the present disclosure.

FIG. 4 is an image summarizing the results of an in vitro experiment in accordance with the present disclosure.

FIG. 5a is an image summarizing the results of an in vitro experiment in accordance with the present disclosure.

FIG. 5b is an image summarizing the results of an in vitro experiment in accordance with the present disclosure.

### DETAILED DESCRIPTION OF THE DISCLOSURE

P. aeruginosa secretes pyocyanin, a blue/green compound that is essential for successful host colonization by P. aeruginosa. Pyocyanin has the ability to oxidize and reduce other molecules and therefore can kill microbes competing against P. aeruginosa as well as mammalian cells which P. aeruginosa has infected. To kill animal cells, pyocyanin must enter the host cells and then interfere with mitochondria functions. Due to the essential nature of pyocyanin in successful host colonization, removal or reduction of its presence within an infected host may successfully treat the overall infection, as P. aeruginosa will struggle to survive without it. P. aeruginosa also secretes siderophore pyoverdine, a compound that provides crucial nutrients and regulation to P. aeruginosa. As with pyocyanin, due to the essential nature of siderophore pyoverdine, removal or reduction of its presence within an infected host may successfully treat and eliminate P. aeruginosa.

The inventors of the present application have determined that certain thermally activated clay minerals can effectively neutralize pyocyanin and siderophore pyoverdine by adsorbing the pyocyanin and siderophore pyoverdine and physically separating the secretions from the host cells, thereby preventing or eliminating infections. These thermally activated clay minerals have beneficial surface properties and porosity that are particularly adapted to adsorbing pyocyanin and siderophore pyoverdine without affecting the host. The inventors of the present application have also determined that these certain thermally activated clay minerals regulate the production and virulence gene expression of pyocyanin and siderophore pyoverdine. In fact, the thermally activated clay regulates the expression of over 50 genes in P. aeruginosa, including those involved in pyocyanin production, siderophore pyoverdine production, biofilm formation, and quorum sensing.

Clay minerals are hydrous aluminum phyllosilicates, which may contain variable amounts of iron, magnesium, alkali metals, alkaline earths and other cations. Clay minerals exist in nature but have to be further processed for them to possess the chemical or physical properties necessary for them to be useful. This processing may include both physical and chemical treatments. Clay directly obtained from earth may contain a multitude of other non-clay minerals (e.g., top soil, quartz, silica, etc) associated with it. However, crushing, sieving (about 20 to about 400 mesh size), sizing (about 1 to about 100 μm particle size or from about 20 to about 50 μm), thermal processing (about 100 to about 800 degrees Celsius), wet-processing, chemical treatment, ion-exchanging, functionalization, and such treatment may impart desired properties to the clay mineral that will impart specific properties that lead to toxin binding catalysis, adsorption, etc.

The clays used in this invention are clays that have been mechanically processed and thermally treated. In one embodiment, the clays are thermally processed and advantageously heated to a temperature between about 100 to 800 degrees Celsius (for example, about 400 to about 800 degrees Celsius) and ground to a fine particle size (e.g., to a particle size of approximately between about 10 microns to as large as about 500 microns or advantageously between about 20 and about 50 microns)(“thermally activated clays”). Methods to make thermally activated clays are well known to a person of ordinary skill in this art. Non-limiting examples of clays which may be processed are: clay minerals, such as smectites (which include montmorillonite, nontronite, beidellite and saponite); alumino-silicate, sepiolite, phyllosilicates; attapulgite (palygorskite); bentonite (e.g., sodium bentonite); hormite, kaolin; and fuller's earth.

In some embodiments the clay may be heated to about 100° C., about 125° C., about 150° C., about 175° C., about 200° C., about 225° C., about 250° C., about 275° C., about 300° C., about 325° C., about 350° C., about 375° C., about 400° C., about 425° C., about 450° C., about 475° C., about 500° C., about 525° C., about 550° C., about 575° C., about 600° C., about 625° C., about 650° C., about 675° C., about 700° C., about 725° C., about 750° C., about 775° C., about 800° C., about 825° C., about 850° C., about 875° C., about 900° C., about 925° C., about 950° C., or about 1000° C. It may be heated for 1 minute up to 12 hours or between about 1 to about 4 hours. Heating may be done statically in a muffled furnace or dynamically in a flash dryer.

In some embodiments of the present invention, the thermally activated clays are montmorillonite clay, attapulgite clay, or hormite, or sodium bentonite, which have been heat treated at a temperature between about 100 to about 800 degrees Celsius.

Non-limiting examples of thermally activated clays are heat treated clays, such as heat treated montmorillonite clays, which have been heat treated at a temperature of between about 100° C. to about 800° C. and have an average particle size between about 32 microns to about 36 microns, such as, for example, Calibrin®-A, Calibrin®-TQ or Calibrin®-Z.

In some embodiments, the present invention may use ion-exchanged or functionalized clays. An “ion-exchanged clay” is a thermally activated clay, such as one of those identified above, that has been reacted with an ion-exchange material. Processes to prepare ion-exchanged clays are well known to one of ordinary skill in this art (for example, D. Carrol, Geological Society of America, 1959, 70(6): 749-779) and processes to prepare these clays are described in more detail below. Generally, the clay is dispersed and stirred aggressively in a salt solution, which contains the cation to be exchanged (e.g. CuCl₂), at a fixed temperature for a fixed amount time. During this process, the naturally present cations in the clay interlayer exude out of the structure and the cations from the salt solution (Cu²⁺) take their place in the clay structure. The clay thus formed may impart different properties than the parent clay due to the presence of different cations (e.g., copper ions) in its structure.

Non-limiting examples of ion-exchanged clays include aluminum, copper or proton exchanged montmorillonite clay; e.g., H-montmorillonite, Al-montmorillonite and Cu-montmorillonite. Non-limiting examples of these clays include copper exchanged Calibri®-A or copper exchanged Calibrin®-Z, aluminum exchanged Calibrin®-A or aluminum exchanged Calibrin®-Z, or proton exchanged Calibrin®-Z.

A “functionalized clay” is a thermally activated clay in which chemical functionalities or an active and specific organic group has been added to the surface of the clay to enhance specific properties of the thermally activated clay or hybrid. Hybrid refers to the formation of a new material containing both inorganic and organic functionalities and are also called hybrid materials. Hybrid materials can exhibit both inorganic and organic properties; e.g., a polymer infused clay is a hybrid which will exhibit the flexibility of a polymer (organic property) and the strength of a clay (inorganic property). A functionalized clay is obtained by reacting a modified clay, such as those heat treated clays identified above, with an amino acid (e.g., histidine or isoleucine), protein (e.g., lysozyme, peptides, etc.). Processes to functionalize clays are well known to one of ordinary skill in the art and processes to prepare these clays are described below.

Non-limiting examples include Calibrin®-A-histidine, Calibrin®-A-isoleucine, Calibrin®-A-lysozyme, or attapulgite-lysozyme.

In some embodiments, the thermally activated clay in ion-exchanged modified clay or a functionalized modified clay is heat treated montmorillonite clay, attapulgite clay, or hormite, or sodium bentonite.

In the above discussion “montmorillonite clay” refers to a clay which is at least 50% montmorillonite, such as the clay found in the Porter's Creek Formation, which is mined in Mississippi, Illinois, Missouri, and Tennessee. Clay minerals are fundamentally constructed of a tetrahedral silicate sheets and octahedral hydroxide sheets and are classified as 1:1 or 2:1 clays. A 1:1 clay consists of one tetrahedral sheet and one octahedral sheet, e.g., kaolinite. A 2:1 clay consists of an octahedral sheet sandwiched between two tetrahedral sheets, e.g., montmorillonite. The smectite group includes dioctahedral smectites (e.g., montmorillonite, nontronite and beidellite) and trioctahedral smectites (e.g., saponite). The illite group includes the clay micas. Other 2:1 clay types which exist include clays such as sepiolite or attapulgite, these clays have long water channels internal to their structure.

Sorbent minerals are minerals that can absorb or adsorb solids, liquids or gases. Illustrative examples include, zeolites, silica, calcite, illite, volcanic silica, mica, and perlite and combinations of these materials. These materials are mechanically processed and thermally treated. These processes involve increasing or decreasing the drying temperature, time or final moisture content or calcining the material.

The sorbent minerals may be ground to a fine particle size (e.g., to a particle size of approximately between about 1 μm to about 500 μm, more advantageously from about 10 μm-about 400 μm, about 50 μm to about 250 μm or about between 20 and 50 microns. Moreover, the sorbent minerals may be advantageously heated to a temperature between 100-800° C. (for example about 400 to about 800° C.).

In some embodiments the sorbent mineral may be advantageously heated to about 100° C., about 125° C., about 150° C., about 175° C., about 200° C., about 225° C., about 250° C., about 275° C., about 300° C., about 325° C., about 350° C., about 375° C., about 400° C., about 425° C., about 450° C., about 475° C., about 500° C., about 525° C., about 550° C., about 575° C., about 600° C., about 625° C., about 650° C., about 675° C., about 700° C., about 725° C., about 750° C., about 775° C., about 800° C., about 825° C., about 850° C., about 875° C., about 900° C., about 925° C., about 950° C., or about 1000° C. It may be heated for 1 minute up to 24 hours or between about 1 to about 4 hours.

Nanoparticles are siliceous, aluminosilicates or oxides. They include colloidal silica, colloidal zeolites, precipitated and fumed silica. The particle size very from about 5 nm to about 100 nm, and possess a surface area between about 50 to about 500 m²/g. Nanoparticles are created or sourced for this application to replicate the functionality of processed thermally activated clays or non-porous materials in regulating the expression of toxins from a bacterial infection.

This invention contemplates using the inventive methods wherever the targeted bacteria reside. The environments may be in vitro, i.e., placed outside living organisms or in vivo, i.e., placed inside a living organism.

In vitro environments include external surface areas where the targeted bacteria congregate, such as household fixtures, countertops, surgical instruments, food processing equipment, food packaging equipment, food packaging, food products, including agricultural products, such as seeds, fruits and vegetables, or processed foods. For agricultural products, the environment might be on the seeds, fruits or vegetables, on the crops plants or in the field (including the soil) where the crops or plants are being cultivated. Similarly, the environment may be processed foods or places where such foods are processed. Moreover, environments include places where animals are raised or reside, such as aqueous environments for raising fish or animal bedding. Other in vitro environments include drinking water for animals (including humans), activated sludge or other areas in the treatment of waste.

The thermally activated clay may be formulated as a solid, liquid, topical solution, or spray.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. The thermally activated clay can be (micro)encapsulated and further formed into a suspension or solid formulation; alternatively the entire formulation can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulations.

Sprayable formulations are typically suspended in a suitable medium before spraying. Such liquid and solid formulations are formulated to be readily diluted in the spray medium, usually water. Spray volumes depend upon the environment being treated and the determination of the spray volume is well within the skill level of one of ordinary skill in the art.

For example, in agriculture applications, the spray volumes can range from about from about one to several thousand liters per hectare, but more typically are in the range from about ten to several hundred liters per hectare. When the sprayable formulations are for agriculture application, the formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic update.

The amounts of thermally activated clay may comprise 0.25% by weight for preventative treatment in an aqueous solution. Additional formulation adjuvants include inert diluents or carriers and surfactants.

Solid diluents are well known to one of ordinary skill in this art and can include, for example, gypsum, titanium dioxide, zinc oxide, starch, sugars (e.g., lactose, sucrose) urea, calcium carbonate, sodium carbonate and bicarbonate, and sodium sulfate.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g, oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation.

The solid and liquid formulations of the present invention may include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyl PEG (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1997.

The thermally activated clay formulations may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.).

As mentioned above, one embodiment of the methods according to this invention is by spraying. The thermally activated clay formulations are also effective by localized application to the locus where the targeted bacteria reside. Methods of contact include application of a formulation of the invention by direct and residual sprays, aerial sprays, gels, seed coatings, microencapsulations, systemic uptake, boluses, aerosols, dusts and many others. The thermally activated clay formulations may also be applied to external surfaces, such as countertops or surgical instruments or food processing equipment, or impregnated materials for fabricating bacterial control devices.

Advantageously, in one embodiment of the present invention, the thermally activated clay does not include any ingredients other than the thermally activated clay itself.

Suitable intervals for the administration of the present invention range from daily to about yearly. Of note are administration intervals ranging from daily or weekly to about once every 6 months. Of particular note are monthly administration intervals. In another embodiment, the present invention are applied for a period of up to 30 days, with some embodiments being 5, 10, or 15 days.

The frequency of applying the present invention to the environment depends upon the nature of the in vivo environment and it is well within the skill level of one of ordinary skill in the art to determine the frequency of applying the thermally activated clay formulation for a particular environment. In one embodiment, the present invention may be applied just once. In other embodiments, the present invention might be applied once or twice a day for a period of time, such as for example, 2, 3, 5, 10, or 15 days or some time period in between.

In vitro environments include areas or places on or inside a living organism, such as an animal (including humans) where the targeted bacteria reside. Animals include, cattle, pigs, lamb, birds (e.g., chickens, ducks, geese and guinea fowl etc.), horses, camels, deer, donkeys, buffaloes, antelopes, rabbits, companion animals (e.g., dogs, cats, rabbits, etc.), rodents, turtles, fish and shellfish (including shrimp and other crustaceans). Areas or places on or inside include, for example, skin surface of a human or animal or is the gastrointestinal tract, nasal passages, urinal tract, vaginal tract, or gut of a human or animal.

The thermally activated clays may be in solid or liquid form. The formulations may contain acceptable carriers comprising excipients and auxiliaries selected with regard to the intended route of administration (e.g., oral, topical or parenteral administration such as injection) and in accordance with standard practice. In addition, a suitable carrier is selected on the basis of compatibility with one or more active ingredients in the formulation, including such considerations as stability relative to pH and moisture content.

Thus, the formulation for human or animal administration may take the form of any pharmaceutically or veterinarily dosage form that would be known to one of ordinary skill in the art, these include controlled-release dosage forms. Solid forms for oral or rectal administration may contain pharmaceutically or veterinarily acceptable binders, sweeteners, disintegrating agents, diluents, flavorings, coating agents, preservatives, lubricants and/or time delay agents. Suitable binders include gum acacia, gelatin, corn starch, gum tragacanth, sodium alginate, carboxymethylcellulose or polyethylene glycol. Suitable sweeteners include sucrose, lactose, glucose or flavonoid glycosides such as neohesperidine dihydrochalcone. Suitable disintegrating agents include corn starch, methylcellulose, polyvinylpyrrolidone, xanthan gum, alginic acid or agar. Suitable diluents include lactose, sorbitol, mannitol, dextrose, cellulose, calcium carbonate, calcium silicate or dicalcium phosphate. Suitable flavoring agents include peppermint oil, oil of wintergreen, cherry, orange or raspberry flavorings. Suitable coating agents include polymers or copolymers of acrylic acid and/or methacrylic acid and/or their esters, and/or their amides, waxes, fatty alcohols, zein, shellac or gluten. Suitable preservatives include sodium benzoate, vitamin E, α-tocopherol, ascorbic acid, methyl parabens, propyl parabens or sodium bisulphate. Suitable lubricants include magnesium stearate, stearic acid, sodium oleate, sodium chloride or talc. Suitable time delay agents for controlled release formulations, include glyceryl monostearate or glyceryl distearate.

Suspensions for oral or rectal administration may further comprise dispersing agents and/or suspending agents. Suitable suspending agents include sodium carboxylmethylcellulose, methylcellulose, hydroxypropylmethylcellulose, polyvinylpyrrolidone, sodium alginate or cetyl alcohol. Suitable dispersing agents include lecithin, polyoxyethylene esters or fatty acids such as stearic acid, polyoxyethylene sorbitol mono- or di-oleate, -stearate or -laurate, polyoxyethylene sorbitan mono- or di-oleate, -stearate or -laurate and the like.

For parenteral administration, including intravenous, intramuscular and subcutaneous injection, a compound of the present invention can be formulated in suspension, solution or emulsion in oily or aqueous vehicles, and may contain adjuncts such as suspending, stabilizing and/or dispersing agents. The thermally activated clay formulation may also be formulated for bolus injection or continuous infusion. Pharmaceutical compositions for injection include aqueous solutions preferably in physiologically compatible buffers containing other excipients or auxiliaries as are known in the art of pharmaceutical formulation. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Formulations for injection may be presented in unit dosage form, e.g., in ampoules or in multi-dose containers. Alternatively, the active ingredient may be in powder form for constitution with a suitable vehicle, e.g., sterile, pyrogen-free water before use.

Formulations for acceptable carriers comprising excipients and auxiliaries selected with regard to the intended route of administration (e.g., oral, topical or parenteral administration such as injection) and in accordance with standard practice. In addition, a suitable carrier is selected on the basis of compatibility with the one or more active ingredients in the composition, including such considerations as stability relative to pH and moisture content.

A pour on formulation may also be prepared for control of parasites in an animal of agricultural value. The pour-on formulations of this invention can be in the form of a liquid, powder, emulsion, foam, paste, aerosol, ointment, salve or gel. Typically, the pour-on formulation is liquid. These pour-on formulations can be effectively applied to sheep, cattle, goats, other ruminants, camelids, pigs and horses. The pour-on formulation is typically applied by pouring in one or several lines or in a spot-on the dorsal midline (back) or shoulder of an animal. More typically, the formulation is applied by pouring it along the back of the animal, following the spine. The formulation can also be applied to the animal by other conventional methods, including wiping an impregnated material over at least a small area of the animal, or applying it using a commercially available applicator, by means of a syringe, by spraying or by using a spray race. The pour-on formulations include a carrier and can also include one or more additional ingredients. Examples of suitable additional ingredients are stabilizers such as antioxidants, spreading agents, preservatives, adhesion promoters, active solubilisers such as oleic acid, viscosity modifiers, UV blockers or absorbers, and colorants. Surface active agents, including anionic, cationic, non-ionic and ampholytic surface active agents, can also be included in these.

The formulations of this invention typically include an antioxidant, such as BHT (butylated hydroxytoluene). The antioxidant is generally present in amounts of at about 0.1-5% (wt/wt). Some of the formulations require a solubilizer, such as oleic acid, to dissolve the active agent, particularly if spinosad is used. Common spreading agents used in these pour-on formulations include isopropyl myristate, isopropyl palmitate, caprylic/capric acid esters of saturated C₁₂-C₁₈ fatty alcohols, oleic acid, oleyl ester, ethyl oleate, triglycerides, silicone oils and dipropylene glycol methyl ether. The pour-on formulations of this invention are prepared according to known techniques. When the pour-on formulation is a solution, the thermally activated clay is mixed with the carrier or vehicle, using heat and stirring if required. Auxiliary or additional ingredients can be added to the mixture of active agent and carrier, or they can be mixed with the active agent prior to the addition of the carrier. If the pour-on formulation is an emulsion or suspension, the formulations can be similarly prepared using known techniques.

In another embodiment, the formulation may be chewable and/or edible product (e.g., a chewable treat or edible tablet). Such a product would ideally have a taste, texture and/or aroma favored by the animal or human to be protected so as to facilitate oral administration.

For oral, subcutaneous or spot-on administration to homeothermic animals, a dose of the thermally activated clay formulation administered at suitable intervals typically ranges from about 2 mg/kg to 800 mg/kg of the animal body weight. For other topical (i.e., dermal) administration, including dips and sprays, a dose typically contains about 2 mg to 800 mg of thermally activated clay per kg of the animal body weight.

Suitable intervals for the administration of the present invention to animals (including humans) range from about daily to about yearly. Of note are administration intervals ranging from about weekly to about once every 6 months. Of particular note are monthly administration intervals (i.e., administering the compound to the animal once every month).

The thermally activated clay formulation may include an inert carrier. The choice of inert carrier depends upon the environment. When the environment is an animal (including a human), suitable inert carriers include water, vegetable oils (e.g., olive oil, peanut or arachis oil, sesame oil, rapeseed oil, palm oil, soybean oil, sunflower oil, safflower oil, or coconut oil), essential oils (e.g., anise oil calamus oil, or cinnamon, oil), aliphatic, aromatic, saturate or unsaturated free fatty acids and their derivatives, liquid paraffin, ethylene glycol, propylene glycol, polyethylene glycol, ethanol, propanol, isopropanol, glycerol, fatty alcohols, triglycerides, polyvinyl alcohol, partially hydrolyzed polyvinyl acetate and mixtures thereof.

In some embodiment of the invention, for oral administration, the pharmaceutical or veterinary composition may be in the form of tablets, lozenges, pills, troches, capsules, elixirs, powders, including lyophilized powders, solutions, granules, suspensions, emulsions, syrups and tinctures. Slow-release, or delayed-release, forms may also be prepared, for example in the form of coated particles, multi-layer tablets or microgranules.

Generally, the thermally activated clay formulation for administration in the method of the invention may be prepared by means known in the art for the preparation of such formulations (such as in the art of veterinary and pharmaceutical compositions) including blending, grinding, homogenizing, suspending, dissolving, emulsifying, dispersing and where appropriate, mixing of the components together with selected excipients, diluents, carriers and adjuvants.

The term “effective amount” as used herein means the amount of a thermally activated clay formulation which regulates the expression of toxins of the bacteria in question. Exemplary ranges for the amounts of thermally activated clay used to treat the bacterial infection are between about 2.5 mg of the thermally activated clay formulation for about every 10,000 colony forming units (cfu) of bacteria.

An “inert carrier” is an inorganic or organic material that does not react with the other components in the thermally activated clay formulation or with the active components loaded onto it. An “inert carrier” may react with components that are not in the thermally activated clay formulation.

The following provides some general methods used to prepare the thermally activated clays.

The clay material is thermally treated at a temperature between 100 to about 800° C. and ground to a fine particle size of between 10 to 800 microns.

In a particular embodiment, the clay material comprises Calibrin® A, which is thermally treated to a temperature between 100 to 500° C. In said embodiment, the bulk density of the Calibrin® A ranges from 341b/ft³ to 531b/ft³ and has a max moisture of 13%. In another embodiment, the clay material comprises Calibrin® Z and thermally treated to a temperature between 350 to 800° C.

The following provides some general methods which may be used to further process clay material.

Amino acid modified clays are prepared by mixing a fixed amount of a clay, such as montmorillonite, into a 1000 ppm solution of an amino acid, such as L-isoleucine or L-histidine and centrifuged, for example, for approximately 30 minutes at about 400 rpm. The solutions are then centrifuged, for example at about 3,500 rpm for 30 minutes, to recover the functionalized clay. The recovered functionalized clay is then successively washed with 500 ml of deionized water to remove any loosely bound amino acids.

Montmorillonite may be ground and washed in deionized water at a ratio of 10 g clay:100 ml water for 24 h under agitation. The resulting clay suspension is then centrifuged and the wash water discarded. The clay is then rehydrated with 100 ml water to which the source for Al³⁺, Cu²⁺, H⁺ cations (e.g., CuSO₄.5H₂O, Al₂(SO₄)₃, HCl, etc.) is then added at an amount of 2 times the CEC of the clay. The resulting slurry is then agitated at 40° C. for 24 h. The ion-exchanged clay is then separated by centrifugation and washed until free from the anions. The washed material is then dried at 105° C., 12 h, and then ground in an agate mortar.

A 500 g of raw montmorillonite clay may be dispersed in 5 L of deionized water with aggressive stirring using an overhead stirrer. The slurry is then passed through a size 350 test sieve (45 μm) by gently rubbing the finger against the screen. The sol is then collected and centrifuged at 3000 rpm for 1 h. The supernatant containing the dispersed clay is then re-centrifuged to separate the heavier fraction once again. The supernatant is then collected and centrifuged once again, and the whole process was repeated for a few more cycles until pure montmorillonite was obtained.

Calibrin® TQ, ultrafine fraction (average size distribution of 10 micrometer particles) of montmorillonite may be prepared using a proprietary alpine or air classification particle separation method.

Approximately 10 g of base clay material (Calibrin®-A or attapulgite) may be placed in a bottle and then 100 mL of water is added. The mixture is then stirred at room temperature for 30 min, to this solution is added 50 mL of lysozyme stock solution (1-10 weight %). The bottle is then capped and then shaken at 250 rpm and 25° C. for 16 hours. After the shaking was completed the mixture is then centrifuged at 5,000 rpm for 30 minutes to collect the lysozyme functionalized clay.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

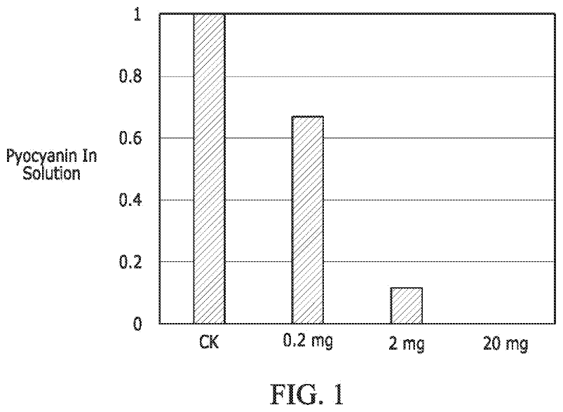

In one in vitro experiment designed to show the binding potential of thermally activated clay to pyocyanin, 1 milliliter of a pyocyanin solution (20 parts per million) was mixed with three different fixed amounts of clay materials: 0.2 milligrams, 2 milligrams, and 20 milligrams. A control sample containing 1 milliliter of the pyocyanin solution was also prepared. In this experiment, CALIBRIN®-A was used as the clay materials, although other clay materials with similar characteristics (i.e., all sheet silicate minerals) are expected to have similar results, including, for example, processed smectite clays (which include montmorillonite, nontronite, beidellite and saponite); alumino-silicate, sepiolite, phyllosilicates; attapulgite (palygorskite); bentonite (e.g., sodium bentonite); hormite, kaolin; and fuller's earth. The mixtures were vortexed for one minute and successively centrifuged at 13,500 revolutions per minute for 10 minutes. The supernatant was analyzed using a spectrophotometry at an optical density of 700 nanometers, and the ratio of absorbed to non-absorbed pyocyanin was calculated by using the measured values of each sample divided by the measured value of the control sample value. As shown in the table at FIG. 1, over 60% of the pyocyanin solution remained in the 0.2 milligram sample, under 20% of the pyocyanin solution remained in the 2 milligram sample, and the pyocyanin solution was completely absorbed by the 20 milligram sample. At the 2 milligram sample, which equates to a ratio of 1:100 of pyocyanin to clay, more than 80% of the pyocyanin is absorbed to clay minerals with a desorption of less than 1%. This demonstrates that clay has high binding affinity for this toxin, such that the pyocyanin binds to the thermally activated clay.

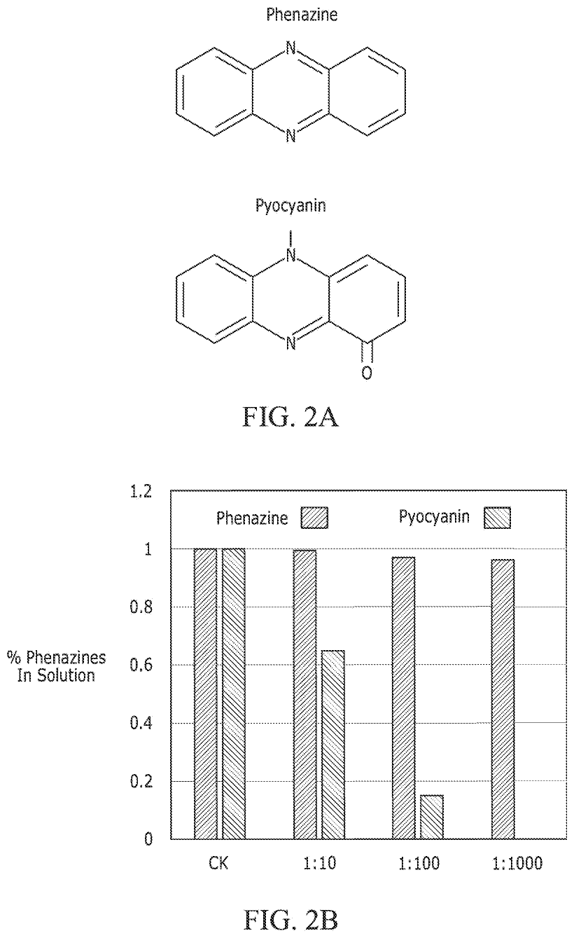

In a second in vitro experiment, 100 micrograms/milliliter of phenzanine or pyocyanin solutions were mixed with varied amounts of CALIBRIN®-A. The mixtures were vortexed for one minute and successively centrifuged at 13,500 revolutions per minute for 10 minutes. The supernatant was analyzed using a spectrophotometry at an optical density of 362 nanometers for phenazine and 700 nanometers for pyocyanin, and the ratio of absorbed to non-absorbed phenazines was calculated by using the measured values of each simple divided by the measured value of the control sample value. To determine whether the sorbent minerals specifically target pyocyanin, but not other related phenazine structures, a pure phenazine solution was used as a control. As shown in FIG. 2a, compared to phenazine, pyocyanin is modified by additions of N-methyl and C-ketone groups. These structural changes alter the physical and chemical properties which are important for adsorption. As shown in the table at FIG. 2b, the majority of phenazine (>95%) remained in the solution (across phenazine to mineral ratios of 1:10 to 1:1000) when treated by varied amounts of sorbent minerals, suggesting that CALIBRIN®-A has a low binding affinity with the core phenazine structure. In contrast, over 60% of the pyocyanin solution remained in the 1:10 (pyocyanin to mineral ratio) sample, and less than 20% of the pyocyanin solution remained in the 1:100 sample, and the pyocyanin solution was completely absorbed by the 1:1000 sample. This demonstrates that modification of phenazine by the N-methyl and C-carbonyl groups alters its properties which allows for strong interactions with thermally activated clay.

In a third in vitro experiment, Vibrio parahaemolyticus was grown in the following cultures using 24 well plate: (1) LB media as a control; (2) LB media and 20 micrograms/milliliter pyocyanin; and (3) LB media, 20 micrograms/milliliter pyocyanin, and 4 milligrams/milliliter CALIBRIN®-A. The ratio of pyocyanin to clay in the third cultures was 1:200. Vibrio parahaemolyticus was used because it is sensitive to pyocyanin and its growth is completely arrested by pyocyanin at a concentration of 20 micrograms/milliliter.

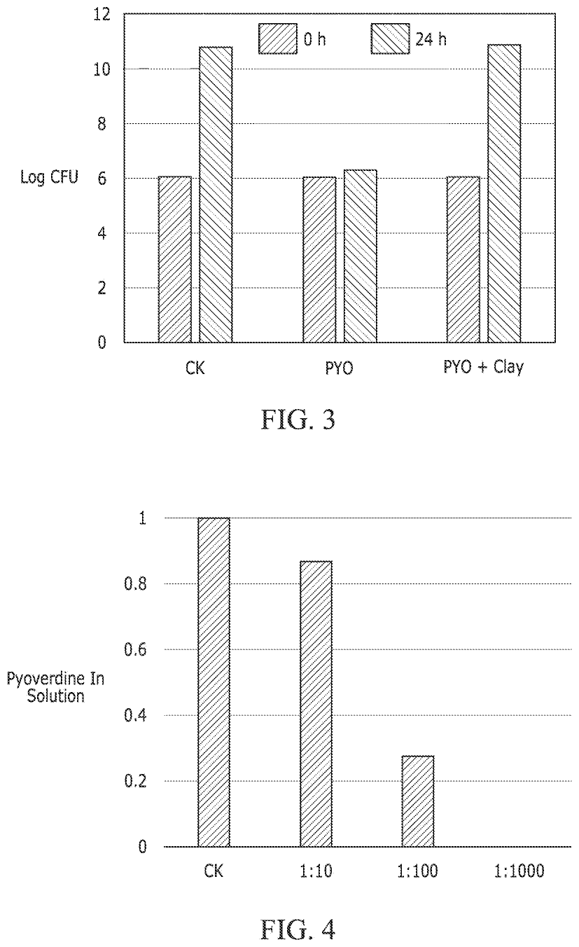

The cultures were incubated at 30 degrees Celsius overnight. Then, bacterial population was determined by serial dilution and counting the colony-forming unit on the LB agar plates. The table at FIG. 3 shows the results of the experiment. CK refers to the control, or first culture referenced above. PYO refers to the second culture with just pyocyanin. PYO+Clay refers to the third culture with pyocyanin and clay. Each culture has two Log CFU measurements, the first (left) referring to the measured amount of Vibrio parahaemolyticus at 0 hours and the second (right) referring to the measured amount of Vibrio parahaemolyticus at 24 hours. As can be seen, the Vibrio parahaemolyticus increased in population in the third culture, demonstrating that the clay absorbed the pyocyanin and neutralized its toxicity to Vibrio parahaemolyticus. The population of Vibrio parahaemolyticus in the third culture also compares favorably with the population of Vibrio parahaemolyticus in the first culture, which contained no pyocyanin to arrest the growth of Vibrio parahaemolyticus. In contrast, the growth of Vibrio parahaemolyticus in the second culture was arrested due to the presence of pyocyanin without any clay to absorb it.

In a fourth in vitro experiment, 10 micrograms/milliliter of pyoverdine solutions were mixed with varied amounts of CALIBRIN®-A. The mixtures were vortexed for one minute and successively centrifuged at 13,500 revolutions per minute for 10 minutes. The supernatant was analyzed using a fluorometer and measuring the fluorescence emission at 460 nanometers during excitation at 400 nanometers. To show the binding potential of thermally activated clay to pyoverdine, 1 milliliter of pyocyanin solution (10 parts per million) was mixed with three different fixed amounts of clay materials: 0.1 milligrams, 1 milligrams, and 10 milligrams. A control sample containing 1 milliliter of the pyoverdine solution was also prepared. While CALIBRIN®-A was used as the thermally activated clay material in this experiment, other clay materials with similar characteristics, such as montmorillonite clays, are expected to have the same results. As shown in the table at FIG. 4, over 80% of the pyoverdine solution remained in the 0.1 milligram sample, under 30% of the pyoverdine remained in the 1 milligram samples, and the pyoverdine solution was completely absorbed by the 10 milligram sample. This demonstrates that the thermally activated clay has a high binding affinity for this compound.

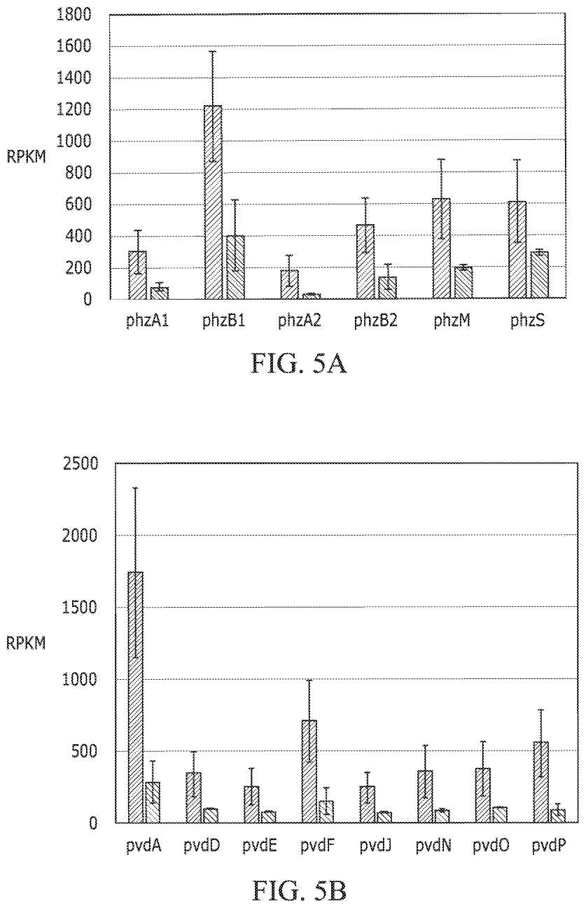

Additional experiments have been performed to examine the regulatory effects of thermally activated sorbent minerals on global gene expression. The transcriptome profiles of culture populations of montmorillonite-treated wild type bacteria were compared to those of the control as ratios of the mean RPKM (Reads Per Kilobase Million) values. Wherein, those for which P<0.05 and differ by more than two-fold are considered differentially regulated. RNA Seq analyses identified a total of 57 genes that were differentially expressed in the presence of sorbent materials compared to the control. A shown in FIG. 5a, 5b, consistent with pyocyanin and pyoverdine production, the expression of pigment biosynthetic genes including: phzA1B1A2B2MS and pvdADEFJNOP was significantly regulated by two to six-fold. The data in FIG. 5a, 5b confirms that sorbent minerals not only adsorb toxins, but also regulate the expression of toxin biosynthetic genes. A pair of quorum sensing genes rhlI/rhlR were down regulated by over twofold. The rhlI/rhlR system is a positive regulator of pyocyanin and deletion of rhlR or rhlI was shown to abolish pyocyanin production.

As shown in Table 1, other genes regulated by sorbent minerals include rahU, clP2 and hsbA.

TABLE 1
Selected Calibrin-regulated genes in P. aeruginosa PAO1

| | | | | |
|---|---|---|---|---|
| | | | Fold | P- |
| Gene ID | Gene | Protein description | change | value |

Quorum sensing

| | | | | |
|---|---|---|---|---|
| PA3476 | rhlI | quorum sensing synthase | −2.60 | 0.02 |
| PA3477 | rhlR | quorum sensing regulator | −2.69 | 0.05 |

Virulence genes

| | | | | |
|---|---|---|---|---|
| PA0122 | rahU | aegerolysin | −2.99 | 0.01 |
| PA3326 | clpP2 | peptidase | −2.50 | 0.04 |
| PA3347 | hsbA | anti-anti sigma factor | −2.11 | 0.05 |

Metabolic genes

| | | | | |
|---|---|---|---|---|
| PA2508 | catC | muconolactone delta-isomerase | 5.80 | 0.00 |
| PA2512 | antA | anthranilate degradation | 3.28 | 0.01 |
| PA2513 | antB | anthranilate degradation | 4.00 | 0.01 |
| PA2514 | antC | anthranilate degradation | 3.83 | 0.01 |

In particular, rahU is an aegerolysin and its expression is controlled by RhlR. Studies have shown that rahU interferes in host cell immunity by inhibiting nitric oxide production and chemotaxis of monocytes and macrophages. Rao, J. et al. RahU: an inducible and functionally pleiotropic protein in Pseudomonas aeruginosa modulates innate immunity and inflammation in host cells. Cell Immunol. 270, 103-113 (2011). The protease clP2 is involved in biofilm formation by activating alginate production. Qiu, D., Eisinger, V. M., Head, N. E., Pier, G. B. & Yu, H. D. ClpXP proteases positively regulate alginate overexpression and mucoid conversion in Pseudomonas aeruginosa. Microbiology 154, 2119-2130 (2008). hsbA is an anti-anti-sigma factor that plays a role in biofilm formation and motility. Valentini, M., Laventie, B.-J., Moscoso, J., Jenal, U. & Filloux, A. The diguanylate cyclase HsbD intersects with the HptB regulatory cascade to control Pseudomonas aeruginosa biofilm and motility. PLoS Genet. 12, e1006354 (2016). A strong feature of the RNA Sequence data set was the upregulation of genes involved in degradation of aromatic compounds by sorbent minerals. Such transcripts included antABC and catC, which were overexpressed between three and six fold. The ant and cat gene products degrade anthranilate, which is a precursor to the quorum sensing molecules. The upregulation of ant and cat genes could result in the decreased synthesis of PQS and therefore reduced expression of multiple virulence factors. It is believed that, this is the first evidence to prove the regulation of a diverse group of virulence genes by thermally activated clay.

Additional experiments have been performed by applicant which indicate that the toxin production of P. aeruginosa is effectively regulated when treated or exposed to a mixture containing a minimum of 0.25% by weight of clay. In contrast, mixtures containing less than 0.25% clay have been less effective. However, this 0.25% clay threshold value may be effected by a multitude of factors, including size of person, age of person, application type, location of infection, severity of infection, etc.

TABLE 2
Impact of clay minerals on pyocyanin and pyoverdine production
of Pseudomonas aeruginosa (PAO1) in vitro. Bacterial
strains were grown in Luria Broth 24 h at 37° C.
without shaking in the presence or absence of Calibrin-
A. The levels of pyocyanin/pyoverdine were measured and
normalized to the No Calibrin-A control.

| Toxin | No Calibrin-A | 0.25% Calibrin-A | 0.12% Calibrin-A |
|---|---|---|---|
| Pyocyanin | 100% | 1% | 42% |
| pyoverdine | 100% | 6% | 58% |

At least two treatment options using thermally activated clay to treat P. aeruginosa are contemplated: topical use for burns and surgical wounds and an enteral formula for systemic infections. The topical treatment option may be in the form of a bandage or cream containing the clay mixture that is intended to be applied to the burned or affected area. The enteral formula may be ingested to treat intestinal tract infections, which is a common reservoir of P. aeruginosa.

While the present disclosure discusses the use of specific thermally-activated clays to treat P. aeruginosa, other clays and even non-clay materials that have similar absorption characteristics as clays may be used in lieu of or in addition to the thermally-activated clays discussed herein. These include, but are not limited to clay minerals, such as smectites (which include montmorillonite, nontronite, beidellite and saponite); alumino-silicate, sepiolite, phyllosilicates; attapulgite (palygorskite); bentonite (e.g., sodium bentonite); hormite, kaolin; and fuller's earth. In addition, the present disclosure and method of treatment of P. aeruginosa may be used with humans as well as chickens, cats, and other animals.

Although the present disclosure and its advantages have been described in detail, it should be understood that various changes, substitutions and alterations can be made herein without departing from the spirit and scope of the disclosure as defined in the appended claims.

Having thus described in detail preferred embodiments of the present disclosure, it is to be understood that the disclosure defined by the above paragraphs is not to be limited to particular details set forth in the above description as many apparent variations thereof are possible without departing from the spirit or scope of the present disclosure.

1. A method for regulating the expression of toxins from a P. aeruginosa bacteria in an environment, which comprises
identifying the toxins of the bacteria which are to be suppressed;
processing a sorbent mineral, chosen from clays, by heat-treatment within a range of about 100 to about 800 degrees Celsius;
preparing a thermally activated clay formulation for administration to the bacterial infection in a particular environment; and
administering an effective amount of thermally activated clay formulation to said environment.

2. The method of claim 1, wherein the effective amount is between 2 milligram to 800 milligram of thermally activated clay per kilogram of the target animal.

3. The method of claim 1, wherein the formulation is configured to be applied topically to a user.

4. The method of claim 1, wherein the formulation is configured to be ingested by a user.

5. The method of claim 1, wherein the toxins of the bacteria are pyocyanin and siderophore pyoverdine secreted by Pseudomonas aeruginosa and the formulation is administered at between a 1:100 and 1:200 pyoverdine/pyocyanin to clay ratio in order to regulate the expression of toxins.

6. The method of claim 1, wherein the thermally activated clay is a processed montmorillonite clay.

7. The method of claim 1, wherein the application of the thermally activated clay modulates the expression of pigment biosynthetic genes including phzA1B1A2B2MS and pvdADEFJNOP.

8. The method of claim 7, wherein the application of the thermally activated clay regulates quorum sensing genes including rhlR and rhlI.

9. The method of claim 8, wherein the application of the thermally activated clay regulates additional virulence genes including rahU, clP2 and hsbA.

10. The method of claim 9, wherein the thermally activated clay reduces the expression of biosynthetic virulence genes by up to six-fold.

11. Thermally activated clays for use in the treatment of a P. aeruginosa bacterial infection wherein the thermally activated clay
is processed from a sorbent material which is heat treated within a range of about 100 to about 800 degrees Celsius;
is prepared for administration to the bacterial infection in a particular environment; and
is administered in an effective amount to said environment.

12. The formulation for use according to claim 10, wherein the effective amount is between 2 milligram to 800 milligram of thermally activated clay per kilogram of the target animal.

13. The formulation for use according to claim 10, wherein the thermally activated clay is administered topically.

14. The formulation for use according to claim 10, wherein the thermally activated clay is configured to be ingested by a user.

15. The formulation for use according to claim 10, wherein the toxins are pyocyanin and siderophore pyoverdine secreted by Pseudomonas aeruginosa and the formulation is administered at between a 1:100 and 1:200 pyoverdine/pyocyanin to clay ratio in order to regulate the expression of toxins.

16. The formulation for use according to claim 10, wherein the thermally activated clay is a processed montmorillonite clay.

17. The formulation for use according to claim 10, wherein the thermally activated clay modulates the expression of pigment biosynthetic genes including phzA1B1A2B2MS and pvdADEFJNOP.

18. The formulation for use according to claim 17, wherein the thermally activated clay regulates quorum sensing genes including rhlR and rhlI.

19. The formulation for use according to claim 18, wherein the thermally activated clay regulates additional virulence genes including rahU, clP2 and hsbA.

20. The formulation for use according to claim 19, wherein the thermally activated clay reduces the expression of biosynthetic and virulence genes by up to six-fold.
